(19) United States
(12) Patent Application Publication
(10) Pub. No.: US 2026/0207273 A1
(43) Pub. Date: Jul. 23, 2026

(54) SURGICAL/MEDICAL INSTRUMENT FOR A SURGICAL ROBOT, AND SUPPORT STRUCTURE FOR SUPPORTINGLY RECEIVING AN END EFFECTOR OF A SURGICAL/MEDICAL INSTRUMENT

(71) Applicant: Aesculap AG, Tuttlingen (DE)

(72) Inventors: Frederick Lenzenhuber, Tuttlingen (DE); Roland-Alois Hoegerle, Tuttlingen (DE); Andre Buerk, Villingen-Schwenningen (DE)

(21) Appl. No.: 19/140,444

(22) PCT Filed: Dec. 19, 2023
(86) PCT No.: PCT/EP2023/086636

§ 371 Date: Jun. 18, 2025

(30) Foreign Application Priority Data
Dec. 19, 2022 (DE) 10 2022 133 861.4

(51) Int. Cl. A61B 34/30; A61B 17/00

(52) U.S. Cl. CPC A61B 34/30; A61B 17/00; A61B 2017/00398; A61B 2017/00477; A61B 2034/305

(57) ABSTRACT

A medical instrument for or of a medical robot includes a support structure for supportingly receiving at least one optionally exchangeable end effector of the medical instrument. A proximal coupling portion is configured for coupling to a distal end segment of the robot. A distal coupling portion is configured for coupling to the end effector. The support structure is or has a connecting component extending in one piece at least between the proximal coupling portion and the distal coupling portion, as a result of which, a coupling-free and/or tolerance-chain-free connection is created between the proximal and distal coupling portions.

### CROSS-REFERENCE TO RELATED APPLICATIONS

This application is the United States national stage entry of International Application No. PCT/EP2023/086636, filed on Dec. 19, 2023, and claims priority to German Application No. 10 2022 133 861.4, filed on Dec. 19, 2022. The contents of International Application No. PCT/EP2023/086636 and German Application No. 10 2022 133 861.4 are incorporated by reference herein in their entireties.

### FIELD

The present disclosure relates to a surgical/medical instrument for a surgical robot, and a support structure for supportingly receiving an end effector of a surgical/medical instrument for a surgical robot.

### BACKGROUND

Robot-assisted handling of surgical/medical instruments is becoming increasingly important in surgery. The advantages are manifold, wherein in particular the minimally invasive use of end effectors, such as tools, HF tips, lighting, optics, cameras, pedicle screws and other work equipment, which can be controlled with high precision and high repeat accuracy, in the area of the procedure on the patient (surgical area) should be mentioned. At the same time, high precision and repeat accuracy, in particular in cases of minimally invasive procedures without visibility in the operating area, are basic prerequisites for performing the procedure with the aid of the robot.

Continuously knowing the position and location of the working point in relation to the surgical site is therefore of crucial importance. For this purpose, means for 3D tracking of the working point are provided on the medical/surgical instrument guided by the robot—‘rigid-bodies’—which can be captured by a 3D camera and can be evaluated via triangulation so that the position and orientation of the working point can be determined. The rigid bodies are also used for initial calibration on site in the operating room. Based on the calibrated initial state, the rigid bodies are tracked-both those of the instrument and, if applicable, those on the patient. If the surgical robot has to be repeatedly moved out of the operating area to change an end effector, a time-consuming recalibration may be necessary after the change. An end effector is basically understood to be any stationary or driven medical/surgical tool, product or aid that can be inserted into the medical/surgical instrument. Examples include a drill, milling cutter, scalpel, HF tip for coagulation, pedicle screw, overview camera, microscope, endoscope, optics, light source, sensor, neurosensor and the like.

Surgical robots have an end effector that is coupled to the surgical instrument. In current systems, the distally arranged end effector is located at the end of a chain of drive and housing components of the medical/surgical instrument. Each of the above components naturally has an ‘intrinsic tolerance’ and also an assembly tolerance in interaction with the neighboring component, resulting in a tolerance chain. The more links (components) there are in the tolerance chain, the more difficult it becomes to maintain a maximum extension (deviation) of the working point-referred to as target zone-within its characteristic specification for the procedure. Such tolerance chains, which result in particular from the number of consecutive assembly and coupling points of the medical instrument, are known, for example, from the ‘Mako’ system from the manufacturer Stryker.

The applicant according to the present disclosure has found that in the known prior art, for example, a first housing part of a medical/surgical instrument is articulated to a mounting interface of a robot via a first coupling, the first housing part is serially connected to further housing parts of the end effector and a receiving chuck for end effectors which is movable relative thereto is mounted on the last housing part, with an internal coupling for receiving an end effector. The applicant has also recognized that such an arrangement makes a general calibration in the operating room necessary in principle. In addition, there is the complex calibration of the surgical instrument in relation to the robot and the enlargement of the target zone with each additional link in the tolerance chain explained above as an example. If the end effector is repeatedly moved out of the operating area for exchanging, this may lead to a decrease in the repeat accuracy of the target zone, so that a time-consuming recalibration that interferes with the operation ultimately becomes necessary.

### SUMMARY

In contrast, one object of the present disclosure is to avoid or at least reduce the disadvantages of the prior art and, in particular, to provide a surgical/medical instrument for a or of a surgical/medical robot via which a position and extension (deviation) of a working point of the end effector are more reproducible. A further object is to provide a support structure, in particular a housing, for supportingly receiving an end effector of a surgical/medical instrument for a or of a surgical/medical robot.

A surgical/medical instrument for a or of a surgical/medical robot comprises a support structure for supportingly receiving an optionally exchangeable, standing or drivable end effector, preferably for supportingly receiving the end effector and a drive of the end effector. The support structure has a proximal coupling portion which is provided and configured for coupling with a distal end segment of the robot. Furthermore, the support structure has a distal coupling portion, which is provided and configured for coupling with the optionally exchangeable end effector. The support structure is or has a connecting component, which extends at least between the proximal coupling portion and the distal coupling portion in one piece, preferably in one piece of material. This establishes a coupling-free and/or tolerance-chain-free connection between the proximal and distal coupling portions.

In the area between the end segment of the robot and the optionally exchangeable/insertable end effector, there are therefore only two coupling points with tolerances due to the one-piece nature of the connecting component. The one at which the proximal coupling portion is coupled with the end segment of the robot and the one at which the end effector is coupled with the distal coupling portion. The tolerance chain and any resulting play are therefore minimal over this area. The position and extent of a working point defined at the tip of the end effector, or the size of the target zone (TZ), thus only depend on the tolerances of the connecting component-and of course of the end segment and of the end effector used. In contrast to state-of-the-art solutions, internal tolerances of the instrument itself no longer have any influence on the position and expansion. Production engineering efforts to achieve a high-precision working point with minimal expansion may thus focus on the production of the connecting component. Provided that the connecting component has been fixed to the end segment and initially calibrated, and that the dimensions and tolerances of the newly inserted end effector are known, it is possible to dispense with recalibration after the change, since only the already known dimensions and tolerances of the new end effector are reintroduced into the system during the change.

Preferably, the target zone according to the present disclosure has a diameter of less than 2 mm to greater than 1 mm.

Particularly preferably, the target zone according to the present disclosure has a diameter of less than 1 mm.

Such small diameters of the target zone may be achieved with a reduced manufacturing effort compared to the state of the art due to the short tolerance chain.

As mentioned above, the term target zone refers to the maximum extension or deviation of the working point. In other words, the term target zone refers to the maximum envelope volume in which the tip of the end effector, i.e. the tool tip, is located due to the tolerance.

For the purposes of the disclosure, the term ‘one-piece’ means at least that—in particular in a force flow—no further coupling portion/coupling point is provided between the proximal and the distal coupling portion. This is preferably implemented in such a way that the proximal and the distal coupling portion are manufactured in one piece of the connecting component, preferably from a continuous material. Alternatively, for example, several pieces of the connecting component may be firmly joined, in particular welded or glued, wherein the coupling portions are then subsequently manufactured on the joined piece.

In principle, an end effector is any medical/surgical tool, product or aid that may be inserted into the medical/surgical instrument, coupled to the medical/surgical instrument, or that is stationary or drivable. Examples include: driven end effectors, such as drills or milling cutters; stationary or fixed end effectors, such as a scalpel, an HF tip for coagulation, an overview camera, a microscope, an endoscope, optics, a light source, a sensor or a sensor arrangement, a neurosensor; products to be used, such as a pedicle screw, or the like. According to the disclosure, the possible type of drive is of course not limited to the above-mentioned rotating configuration, but includes any type of drive commonly used in surgery, such as an oscillating drive.

Preferably, the surgical/medical instrument is equipped with the optionally exchangeable end effector by coupling it to the distal coupling portion (32). Preferably, the surgical/medical instrument comprises a set of optionally exchangeable end effectors of different configuration and/or nominal size, wherein one is inserted and the others are provided.

The tighter the tolerances of the connecting component are manufactured, in particular its proximal and distal coupling portion, the more enhanced are the aforementioned advantages.

Preferably, in a further development, the connecting component has high-precision shape tolerances, alignment tolerances and position tolerances, at least with regard to the coupling portions.

According to a further development, the coupling portions have a fixed alignment, angularity or adjustment in relation to each other. In this way, a main axis or working axis of the end effector is fixed at a predetermined angle relative to the main axis or working axis of the end segment of the robot. Depending on the type of end effector and the requirements for its handling, the connecting component may have a fixed parallel or a fixed angular, in particular right-angled, alignment of the coupling portions to each other. The alignment may be defined via the contact planes of the coupling portions or via their main axes.

In order to be able to align the end effector relative to the end segment without changing the connecting component, the connecting component is provided and configured in a variant for adjusting the contact planes or main axes of the coupling portions relative to each other.

In a preferred further development, the connecting component is a housing or at least a housing portion of the medical instrument, in particular a housing or a housing portion of a drive of the medical instrument that may be coupled to the end effector. In this way, one and the same component can fulfill two functions: on the one hand, the tolerance-chain-free connection of the proximal coupling portion to the distal coupling portion and, on the other hand, the conventional protective function of a housing.

In order to, at least in sections, accommodate and mount the end effector or a driveshaft of the drive and the end effector coupled to the driveshaft, the housing has a shaft housing portion on which the distal coupling portion is configured in a further development. Preferably, the shaft housing portion extends in the shape of a shaft or sleeve. It therefore has a slim design and takes up little installation space.

In order to insert the end effector or the driveshaft and the end effector coupled to it, the shaft housing portion preferably has a proximal inlet opening. Distally, it preferably has an outlet opening through which the end effector passes during the intended operation, i.e. when the end effector is fitted.

As already explained above, the alignment of the coupling portions of the connecting component may be predetermined differently (parallel, angled) in order to optimize the handling of the end effector. In a possible further development, this alignment may be predetermined by shaping the shaft housing portion. In a particularly simple variant, the shaft housing portion extends straight from its inlet opening to the distal coupling portion, resulting in parallelism. Alternatively, it may extend curved in this area, at least in portions, which leads to angularity.

For repeatable and gentle insertion of the end effector into the shaft housing portion, the latter preferably has an insertion aid, preferably in an area of the inlet opening. The insertion aid is preferably configured as a continuous, preferably funnel-shaped tapering of an interior or receiving space of the shaft housing portion. The interior or receiving space preferably extends from the inlet opening to the outlet opening.

According to a further development, the shaft housing portion has a radial constriction or an inner radial collar, from which an axial stop of the distal coupling portion is formed. This axial stop is provided and configured so that a correspondingly shaped axial stop of the end effector is brought into axial contact with it.

The axial contact may be configured directly or indirectly. In the latter case, for example, via at least one axial plain bearing or axial roller bearing, which is placed at the radial constriction or at the inner radial collar.

The axial stop inside the shaft housing portion is easier to manufacture the closer it is configured to the distal end portion of the shaft housing portion. In a preferred further development, the radial constriction or the inner radial collar is therefore formed by a distal end wall of the shaft housing portion and is penetrated by the outlet opening.

In order to be able to minimize the expansion of the working point of the end effector in the radial direction, the shaft housing portion has an inner circumferential surface according to a further development, from which a radial stop of the distal coupling portion is formed. This is provided and configured so that a correspondingly shaped radial stop of the end effector is brought into radial contact with it. The radial contact may be configured directly or indirectly. In the latter case, for example, via at least one radial plain bearing or radial roller bearing that is placed on the inner circumferential surface.

Combinations of plain bearings and roller bearings are possible.

According to a further development, a radial stop and/or axial stop coaxial to the inlet opening is provided proximally on the shaft housing portion and configured to support the end effector or to support a drive shaft of the drive of the end effector.

Components of the drive are, for example, a motor, a gearbox coupled to it and a driveshaft coupled to the gearbox, which in turn may be coupled to the end effector. The use of a gearbox enables, depending on its configuration, an aligned or non-aligned arrangement of the motor relative to the end effector.

In a minimal embodiment, the drive comprises the motor, which is provided and configured for direct coupling with the end effector, preferably with a shaft of the end effector. In particular in the case of a rotatable end effector, this results in the possibility of an aligned arrangement of the motor relative to the shaft housing portion. The aligned arrangement has the advantage that the drive has a narrow configuration. However, it may result in the drive being comparatively long.

In order to minimize the overall length of the drive and its housing, according to one variant, at least one component of the drive is arranged laterally to the shaft housing portion provided. According to a preferred further development, the housing has a motor housing portion lateral to the inlet opening for this purpose, which is provided and configured to accommodate at least the motor.

Preferably, the main axes of the shaft housing portion and the motor housing portion are parallel. In other words: a driveshaft of the motor is parallel to the driveshaft of the end effector accommodated in the shaft housing portion or to the shaft of the end effector. This has the advantage that a simple spur gear may be provided for torque transmission.

Alternatively, the main axes of the shaft housing portion and the motor housing portion may be angled towards each other. In this way, the installation space occupied by the drive may be optimized for use in the operating room.

Preferably, the motor housing portion is essentially cylindrically configured.

According to a further development, the housing comprises a gearbox housing portion provided and configured to accommodate a gearbox of the drive of the end effector at least in portions. The gearbox housing portion may be formed as an independent part or by a portion of the motor housing portion, the shaft housing portion or both.

According to a first variant of the housing, the motor housing portion is connected in alignment with the shaft housing portion. The connection is either direct, i.e. without a gearbox and the corresponding gearbox housing portion, or it is indirect, in that all three housing portions are connected in alignment. As already mentioned above, this enables a comparatively narrow but long configuration of the housing and thus of the medical instrument.

Alternatively, the motor housing portion is connected laterally to the shaft housing portion via the gearbox housing portion. The lateral connection of the motor housing portion to the shaft housing portion has the advantage that it is no longer necessary to dismantle the motor in order to change the end effector, which requires the end effector to be pulled out of the shaft housing portion in a proximal direction. Compared to the aligned arrangement, this variant therefore makes it easier to change the end effector.

According to a further development of the housing for a drive with a gearbox, at least one tapering stepped in the insertion direction is configured in the shaft housing portion, which is provided and configured for the rotatable mounting of a drive element of a gear stage of the gearbox of the drive. The output element may be a friction wheel of a belt transmission or a cogwheel of a cogwheel transmission. It may be mounted directly on the stage or indirectly via a plain bearing or roller bearing.

For a multi-stage configured gearbox, according to a further development, a series of such stepped tapering rings is configured, via which the shaft housing portion is increasingly narrowed in the insertion direction, wherein each tapering is provided and configured to support a drive element of another one of a plurality of gear stages of the gearbox.

Alternatively, the row of such output elements may be arranged in a stack in exactly one radial and stepped tapering of the shaft housing portion.

According to the previous description, one advantage of the connecting components configured according to the disclosure with only two coupling points subject to tolerances is that, under certain conditions, recalibration during the procedure in the operating room may be dispensed with after changing the end effector. One necessary condition is knowledge of the characteristic dimensions and tolerances of the newly inserted end effector. The new position and the new extension of the working point can then be calculated based on the initially calibrated connecting component and the dimensions and tolerances of the new end effector. Recalibration in the OR is no longer absolutely necessary.

In a preferred further development, the instrument therefore has at least one end effector, preferably a set of optionally interchangeable end effectors, which is/are measured and documented with regard to its/their characteristic dimensions and tolerances. This measurement is preferably carried out outside the operating room, preferably in the factory in the course of producing the end effector, in particular in the course of its manufacture, so that the dimensions and tolerances can be tightened with the insertion or coupling of the respective end effector into or with the instrument by a control unit which is configured to calculate the position and the extension of the working point. In this way, the new position and the new extension of the working point can be calculated after insertion/coupling of the end effector and recalibration is not necessary. The effort of measuring or calibrating on site in the operating room is thus eliminated in this way and-as already mentioned-is instead located at the factory, which represents a major advantage over the state of the art.

In order to be able to determine these dimensions and tolerances for each insertable end effector, or to be able to tighten them as mentioned above, the surgical/medical instrument, preferably the connecting component, preferably the housing, has, in a preferred further development, a detection unit which is configured so that at least one ID and/or the characteristic properties and/or the dimensions and tolerances of the end effector which passes the detection unit, preferably a sensor, during insertion may be read out via it. The prerequisite for this is preferably that the insertable end effectors have a corresponding emitter or a corresponding tag on which the ID or the aforementioned data is or are stored.

Preferably, the detection unit is configured as a sensor, in particular as an NFC antenna for reading an RFID tag of the end effector. The data stored in the RFID tag are preferably the article number, serial number, sort, type and nominal size of the end effector as well as in particular its characteristic dimensions and tolerances, in particular the characteristic distance of the working point from the axial stop of the end effector, the nominal size of the end effector, its concentricity, its coaxiality and the like. Preferably, the detection unit is arranged in an area of the inlet opening or the insertion aid described above. Preferably, the detection unit has a receiving capacity which covers at least one cross-section of the shaft housing portion, preferably the inlet opening or the insertion aid. Preferably, the detection unit extends over the entire shaft housing portion or it extends at least partially over the entire shaft housing portion, preferably evenly distributed.

In a preferred embodiment, the proximal coupling portion of the connecting component is straight and beam-shaped or it is configured in the shape of a pincer or clamp. Depending on the configuration, other advantageous coupling movements result for attaching the connecting component to the end segment of the robot.

In order to define the position and orientation of the proximal coupling portion relative to the end segment in the simplest way, the proximal coupling portion has, in a preferred embodiment, three point-like, proximal coupling elements, of which a proximal coupling plane is spanned. The three coupling elements are provided and configured in particular for engagement with three coupling elements on the end segment side that are adapted to them. Preferably, the arrangement of the three coupling elements forms corners of an equilateral triangle.

According to a possible further development, at least one of the proximal coupling elements has a centering bore or trough and another has a centering pin or centering sphere.

According to a possible further development, at least one of the proximal coupling elements has a stop acting transversely to the proximal coupling plane.

Due to the configuration according to the disclosure, the connecting component extends in one piece up to the distal stop, i.e. close to the working point of the end effector. This makes it easy to guide supply ducts into this area. According to an advantageous further development, the connecting component has at least one such channel which leads distally or which opens distally, preferably at a distal end face and/or a distal outer surface of the connecting component, in particular of the shaft housing portion. The channel(s) preferably extend(s) in and along a wall of the shaft housing portion. The respective channel may be provided and configured to accommodate a light guide for illuminating a surgical area, an optical system for viewing the surgical area, a coolant for cooling the end effector or its bearing, a data line for a distally mountable camera or a distally mountable sensor, in particular a force sensor for detecting forces and/or moments of the working end, or it is provided and configured for extracting fluid in the surgical area.

Preferably, the connecting component has a fastening interface that is configured so that a drape may be attached to it.

In a preferred embodiment, the connecting component is a sterile product.

The drive may be manual or motorized, or the drive may be manual and servomotor-assisted.

In a manual or servomotor-assisted configuration, the drive has a handpiece. In the manual configuration, the handpiece is preferably provided and configured to be coupled directly to the end effector. In the servomotor-assisted embodiment, it is preferably provided and configured to be coupled to a specific gear stage of the gearbox.

In a preferred embodiment, a gearbox of the drive has several gear stages. The drive is particularly flexible in terms of speed and torque if it has several gear stages. Preferably, each of the gear stages is assigned an output element that is mounted axially and circumferentially on a stepped tapering of the shaft housing portion and is rotatable.

For simple coupling of one of the output elements with an end effector assigned to it, in particular with its shaft, the output elements have a, preferably central, passage recess with an internally circumferential coupling portion.

A particularly simple assignment and coupling of the respective gear stage with an end effector assigned to it is made possible if the inner diameters of the passage recesses decrease in steps in the insertion direction according to a further development.

The drive preferably has a set of interchangeable optionally usable end effectors, each of which is configured with a shaft of the same length. The end effectors each have an outer circumferential coupling portion at the same height—measured from the coupling portion of the end effector—which is provided and configured for coupling with one of the inner circumferential coupling portions.

The precise coupling of a specific end effector with the gear stage specifically assigned to it, i.e. with the specific output element, is easily achieved if the outer circumferential coupling portions each have a unique tuple of outer diameter and length according to a further development: the largest of the outer diameters is assigned the smallest length and the smallest of the outer diameters is assigned the largest length. The outer diameters in between decrease in steps in the insertion direction, while the lengths increase in steps.

### BRIEF DESCRIPTION OF THE DRAWINGS

FIG. 1 shows a surgical robot with a surgical instrument coupled to it according to the state of the art, in perspective view;

FIG. 2 shows a connecting component, configured as a housing, of a surgical/medical instrument of a surgical robot according to a first configuration example, in perspective view;

FIG. 3 shows the housing according to FIG. 2 in a partial perspective section;

FIG. 4 shows a drive with the housing according to FIGS. 2 and 3 according to a first configuration example, in perspective view;

FIG. 5 shows the housing according to FIGS. 2 and 3 in a longitudinal section;

FIG. 6 shows a connecting component, configured as a housing, of a surgical/medical instrument of a surgical robot, according to a second configuration example, in a longitudinal section;

FIG. 7 shows the housing according to FIG. 6 in a proximal view;

FIG. 8 shows a partial section of the housing shown in FIG. 5 with the end effector inserted;

FIG. 9 shows a drive of a surgical/medical instrument according to a second configuration example, in a longitudinal section;

FIG. 10 shows a drive of a surgical/medical instrument according to a third configuration example, in a longitudinal section;

FIG. 11 shows a drive of a surgical/medical instrument according to a fourth configuration example, in a longitudinal section;

FIG. 12 shows the same end effector with different coupling portions for coupling with different gear stages of a drive of the surgical/medical instrument;

FIG. 13 shows a detail of the drive according to FIG. 11, in perspective view;

FIG. 14 shows the drive as shown in FIG. 13 with the end effector inserted, in a perspective view;

FIG. 15 shows a drive with the housing according to FIG. 5, according to a fifth configuration example, in a longitudinal section;

FIG. 16 shows the drive according to FIG. 15 with an end effector to be inserted;

FIG. 17 shows a drive of a surgical/medical instrument according to a seventh configuration example and with the housing according to FIGS. 6 and 7, in a longitudinal section; and

FIG. 18 shows the drive according to FIGS. 15 and 16 with channels running in the housing.

### DETAILED DESCRIPTION

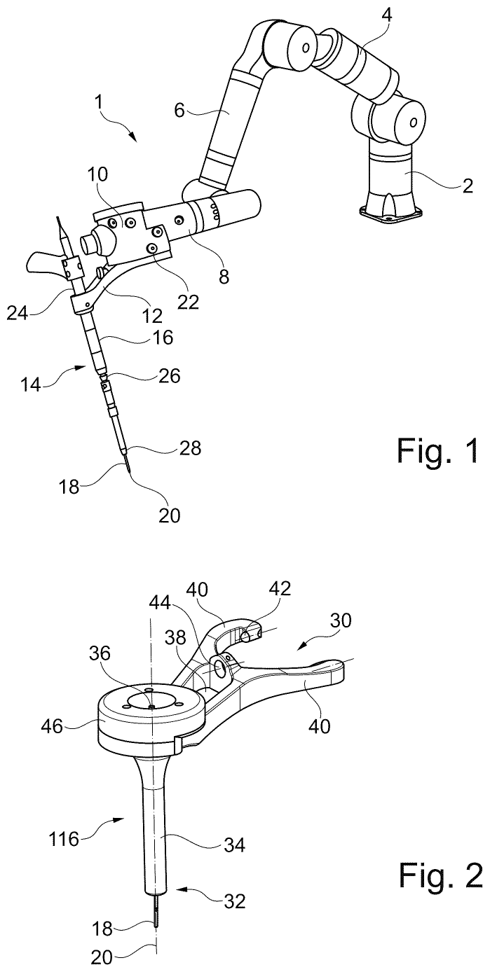

FIG. 1 shows a robotic arm of a surgical robot 1 with a drive of an end effector 18 with a housing 16 according to the state of the art. The robot arm has segments 2, 4, 6, 8, 10 connected in an articulated manner and an end segment 10. A surgical instrument 14 is coupled to the end segment 10 via a coupling arm 12. The instrument 14 is fixed in a receptacle of the coupling arm 12 with its housing 16. A working end or end effector 18 of the instrument 14 emerges from the distal end of the housing 16. The working point 20 of the instrument 14, which is effective in the surgical area, is located at its tip. Starting from the end segment 10 to the end effector 18, the instrument 14 has a chain of coupling points 22, 24, 26, 28. In the system shown in FIG. 1, components of the drive of the end effector 18 and of the housing 16 are connected between the end segment 10 and the end effector 18 via an abundance of coupling points 22, 24, 26, 28. This creates a tolerance chain and therefore also a play, which increases the spherical expansion of the working point, the target zone. This effect has to be minimized or eliminated.

For this purpose, a, preferably high-precision, connecting component for the medical/surgical instrument is proposed, the configuration examples of which are described below in FIGS. 2 to 18. In the configuration examples, the connecting component is configured as a housing-like component and is hereinafter referred to as ‘housing’ for the sake of simplicity. It is characterized in particular by the absence of several coupling points, which minimizes the tolerance chain. The target zone of the end effector 18 can thus be greatly narrowed and optimized.

FIG. 2 shows a perspective view of a connecting component configured as a housing 116 for a medical/surgical instrument comprising the end effector 18, according to a first configuration example. The housing 116 has a proximal coupling portion 30, which may be coupled to the end segment 10 (cf. FIG. 1), and a distal coupling portion 32, to which the end effector 18 is coupled. The housing 116 extends between the coupling portions 30 and 32 in one piece, i.e. without any further coupling points in the frictional connection between the end segment 10 and the end effector 18. The number of coupling points is thus reduced to a minimum of ‘two’. Compared to the solutions of the prior art, internal coupling points of a drive of the end effector 18 or otherwise existing coupling points of the housing 116 therefore have no influence on the position of the working point 20. Only the tolerances of the coupling portions 30 and 32 determine the target zone. In order to minimize the expansion of the target zone, the coupling portions 30 and 32 are therefore manufactured with high precision in terms of shape, alignment and position tolerances.

According to FIG. 2, the housing 116 has a tubular or sleeve-shaped shaft housing portion 34, which is penetrated by a proximal coupling 36 of the end effector 18. The coupling arm 12 is attached laterally to the shaft housing portion 34 at an obtuse angle. The coupling arm 12 is weight-optimized via a recess 38. The coupling portion 30 branches out into two coupling legs 40, on each of which a spherical coupling element 42 (one is concealed) of the coupling portion 30 is configured radially on the inside. At the apex of the branching point, the coupling portion 30 has a further coupling element 44, which is configured as a through bore and opens in the direction of the recess 38. Here, the advantage according to the disclosure lies in the precision of the relative position of the coupling plane spanned by the coupling elements 42, 44 to the coupling plane spanned by the coupling portion 32.

According to FIG. 2, the housing 116 also has a gearbox housing portion 46, which is described further below.

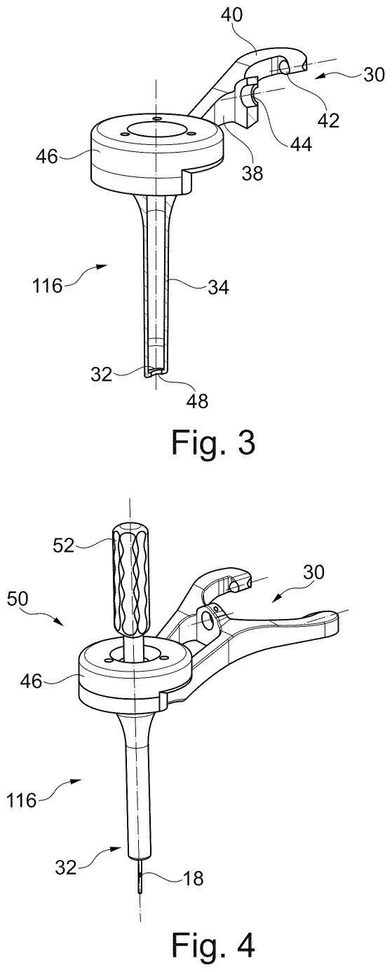

FIG. 3 shows the housing 116 according to FIG. 2 in a longitudinal section shown in perspective without the end effector 18. The gearbox housing portion 46 is shown uncut. The distal coupling portion 32 arranged on the inside is visible and the sleeve-shaped extension of the shaft housing portion 34 can be seen. The distal coupling portion 32 has an axial stop in the extension direction of the end effector 18, which in the configuration example shown is formed by a radial narrowing of the shaft housing portion 34 on the end face. This constriction borders an outlet opening 48 of the shaft housing portion 34, through which the end effector 18 (cf. FIG. 1) exits.

FIG. 4 shows a manual, servomotor-assisted drive 50 with the housing 116 according to FIGS. 2 and 3. The drive 50 has a handpiece 52, which may be used to initiate rotation of the end effector 18. The handpiece 50 is configured to be mountable and is in operative connection within the gearbox housing portion 46 with a gearbox insert of the drive 50, which is not shown. The rotation of the handpiece 52 thus causes the server motor support. In this way, for example, the screwing in of a pedicle screw may be motor-assisted and the surgeon still retains a feel for the torque applied.

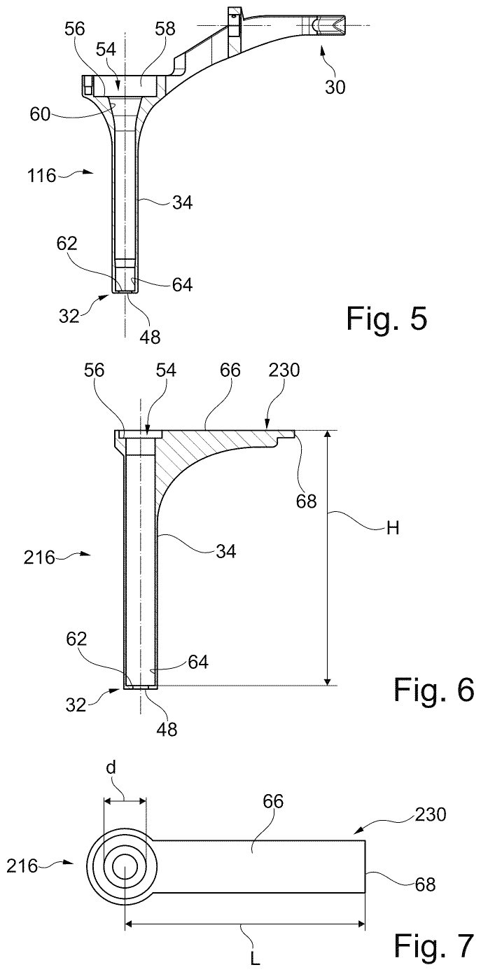

FIG. 5 shows the housing 116 in a longitudinal section. The gearbox housing portion 46 is not shown here. In addition to the FIGS. 2 to 4, a proximal inlet opening 54 of the shaft housing portion 34 is visible, through which the end effector 18 can be inserted into the housing 116. The inlet opening 54 is comparatively wide. In the insertion direction, the shaft housing portion has a comparatively strong, stepped tapering 56 in the vicinity of the inlet opening 54. This forms a cylindrical receiving space 58 for a set of output elements of the gearbox (not shown). The stepped tapering 56 is adjoined by a funnel-shaped tapering 60, which acts as an insertion aid when the end effector 18 is inserted. At the distal end of the shaft housing portion 34, an axial stop 62 and a radial stop 64 of the distal coupling portion 32 are shown in section. The former is a high-precision machined, inward-facing annular front surface that surrounds outlet opening 48, while radial stop 64 is a portion of an inner surface of shaft housing portion 34 adjacent to the annular front surface.

FIGS. 6 and 7 show a second embodiment example of a housing 216 of an end effector of a surgical instrument of a surgical robot, wherein only the differences to the first configuration example will be discussed in order not to overload the text. In contrast to the housing 116 according to FIGS. 2 to 5, the shaft housing portion 34 extends from its inlet opening 54 to the outlet opening 48 with only one radial tapering 56. The insertion aid (cf. 60, FIG. 5) has been dispensed with here. The step-shaped, radial tapering 56 of the housing 216 is kept comparatively flat. The tapering 56 is therefore suitable for holding only one drive element of a gear stage.

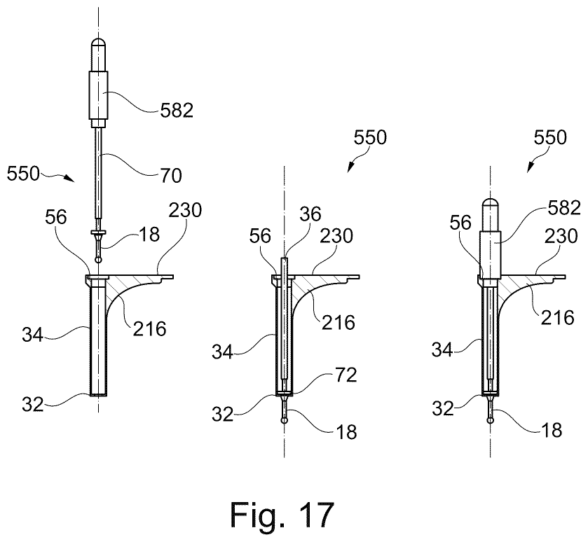

Alternatively, it serves as a proximal bearing point for a shaft of the end effector or of a drive motor to which this shaft is coupleable, which is explained further below in FIG. 17. In the configuration example shown, the proximal coupling portion 230 of the housing 216 has an axial coupling surface 66 for frontal contact with a corresponding coupling surface of the end segment 10 and a lateral coupling surface 68 for lateral contact with a corresponding coupling surface of the end segment 10. According to FIGS. 6 and 7, the dimension H, which defines the distance between the axial coupling surface 66 of the proximal coupling portion 230 and the axial stop 62, the dimension L, which defines the distance between the lateral coupling surface 68 of the proximal coupling portion 230 and the center of the radial stop 64, and the dimension d, which defines the diameter of a radial stop in the area of the inlet opening 54, are manufactured with high precision with respect to shape, alignment and position tolerance.

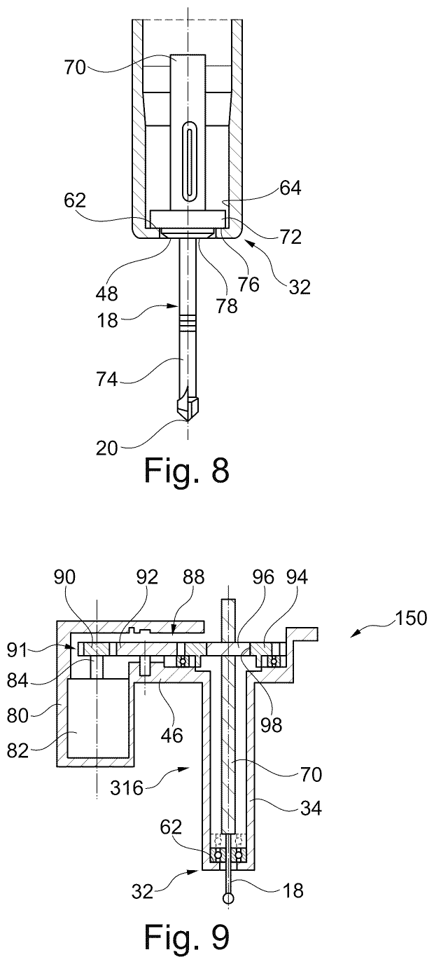

FIG. 8 shows the distal coupling portion 32 of the respective shaft housing portion 34 of FIGS. 2 to 7 in detail, with coupled end effector 18. This has a shaft 70 extending within the shaft housing portion 34, a radial collar 72 adjoining it distally and a working end 74 emerging from the outlet opening 48 with the working point 20 at the tip. It can be clearly seen that a distally oriented annular front surface 76 of the radial collar 72 is in sliding contact with the axial stop 62 of the distal coupling portion 32. The end effector 18 can be centered either via a circumferential surface of the radial collars 72, which is supported on the inner surface forming the radial stop 64, or via a partially conical centering collar 78, which is immersed in the outlet opening 48.

As already mentioned above, according to the present disclosure, the target zone with a diameter of less than 2 mm, particularly preferably less than 1 mm, can be achieved with little manufacturing effort, which is due to the fact that fewer surfaces have to be machined due to the shortened tolerance chain than in solutions according to the prior art. Irrespective of the respective configuration example according to the disclosure, the term target zone, with reference to FIG. 8, means an enveloping volume/tolerance space extending around the tip of the end effector 18, in which the tip of the end effector 18 is arranged according to the tolerance after assembly of the end effector 18.

FIG. 9 shows a drive 150 of an end effector of a surgical robot according to a second configuration example, in a longitudinal section. In contrast to the previous description, the housing 316 has a motor housing portion 80 connected laterally to the shaft housing portion 34, in which a drive motor 82 with a drive shaft 84 parallel to the shaft 70 is accommodated. The housing portions 34 and 80 are connected via a gearbox housing portion 46, in which a gearbox 88 configured as a spur gear is accommodated in sections. The gearbox 88 has a drive spur gear 90 coupled to the driveshaft 84 and an output spur gear 94 coupled to the shaft 70 and non-rotatably connected to it via a cogwheel 92. The cogwheels 90, 92 and 94 form a gear stage 91 with a fixed transmission ratio of the drive 150. For coupling, the shaft 70 of the end effector 18 has an outer circumferential coupling portion 96 in rotationally fixed connection with an inner circumferential coupling portion 98 of a passage recess of the output spur gear 94. In contrast to the previous configuration examples, the end effector 18 is not supported via a radial collar, but via an annular front surface 76 of the shaft 70 and two ball bearings on the axial stop 62 of the distal coupling portion 32.

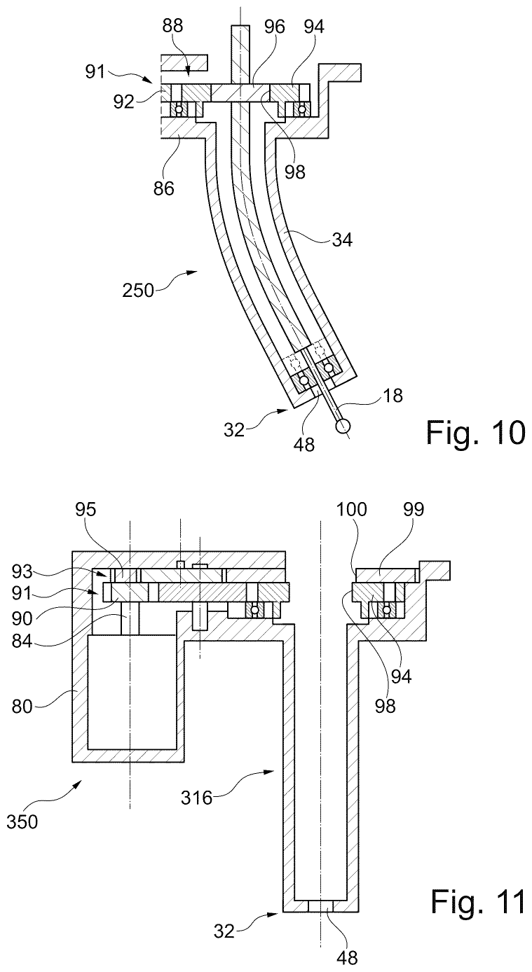

FIG. 10 shows a drive 250 of an end effector 18 of a surgical robot according to a third configuration example, in a longitudinal section. In contrast to the configuration example according to FIG. 9, the shaft housing portion 34 does not extend linearly, but curved. Shaft housing portion 34 is rigidly configured and adapted to a specific requirement in the surgical area via the curvature. It is necessary here that the shaft 70 is flexible.

FIG. 11 shows a drive 350 of an end effector 18 of a surgical robot according to a fourth configuration example, in a longitudinal section. Deviating from the configuration example according to FIG. 9 or 10, a second gear stage 93 is provided in addition to the first gear stage 91. Like the drive spur gear 90 of the first gear stage 91, its drive spur gear 95 is firmly coupled to the driveshaft 84. Both spur gears 90 and 95 therefore always rotate with the driveshaft 84 and drive their respective output spur gears 94 (first gear stage 91) and 99 (second gear stage 93).

This results in different speeds at the drive spur gears 94 and 99. An end effector 18 that is coupleable to the respective inner circumferential coupling portion 98, 100 may thus be driven at different speeds, depending on which coupling portion 98, 100 it engages with.

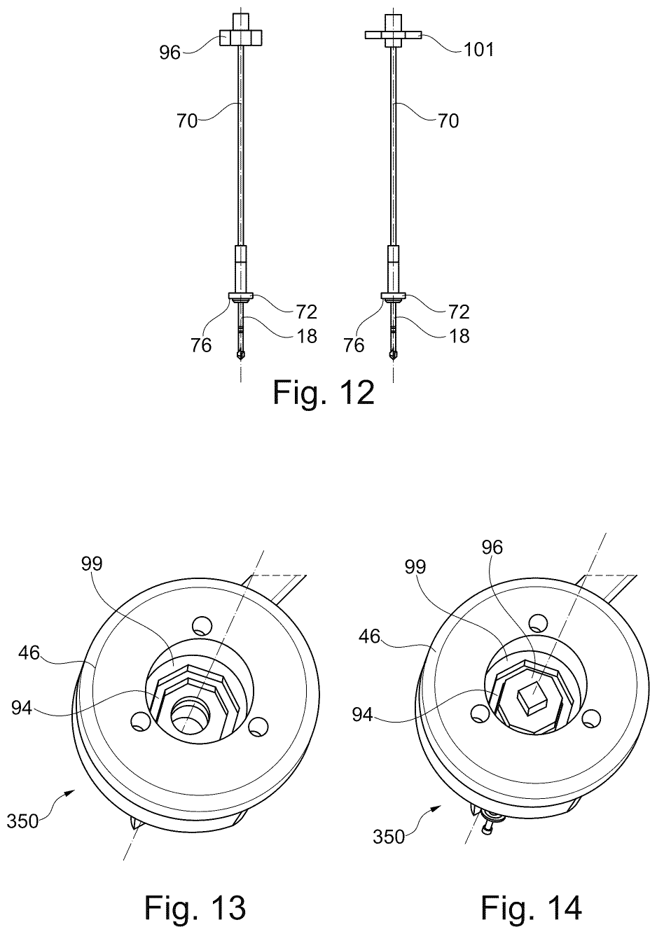

FIG. 12 illustrates this possibility of providing the end effector 18 with different external circumferential coupling portions 96, 101, bringing them into engagement and thus driving them at different speeds. Depending on the outer circumferential coupling portion 96, 101 used as shown in FIG. 12, the end effector 18 is driven by the first gear stage 91 at its own speed n1 or by the second gear stage 93 at its own speed n2.

FIG. 13 shows the drive 350 as shown in FIG. 11 in the area of the drive track gears 94 and 99 in a perspective view from above.

FIG. 14 shows the drive according to FIG. 13, wherein the end effector 18 on the left in FIG. 12, which has the outer circumferential coupling portion 96, is inserted. Due to its smaller diameter and greater height, the coupling portion 96 only couples with the lower, inner circumferential coupling portion 98 of the output spur gear 94 of the first gear stage 91. The second gear stage 93 thus remains disengaged and only co-rotates, i.e. without torque transmission to the end effector 18.

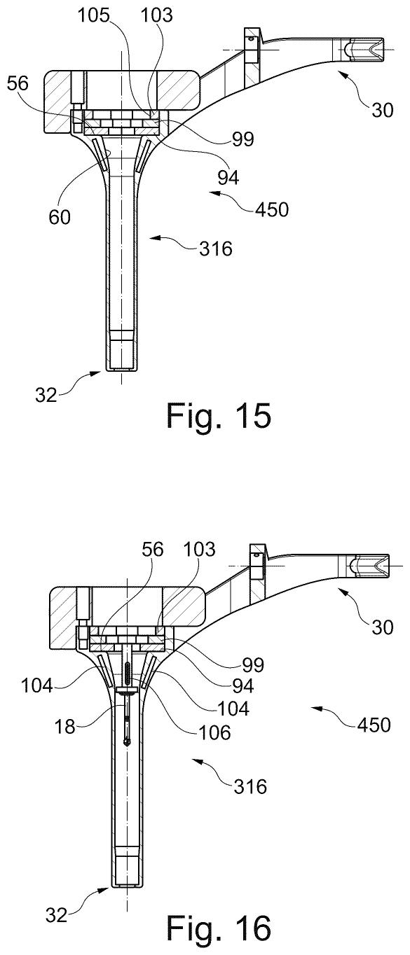

FIG. 15 shows a drive 450 which differs from the one shown in FIGS. 11, 13 and 14 only in its higher number of gear stages—three instead of just two. This may be seen from the three-layered set of drive spur gears 94, 99 and 103, wherein the drive spur gears 94, 99 represent the first and second gear stages 91 and 93 described above and the output spur gear 103 represents the additional third gear stage.

FIG. 16 shows a further detail of the drive 450, which makes it possible to uniquely identify the inserted end effector 18 and to transmit its ID or other characteristic data records to a control unit of the surgical robot. This is an RFID antenna or an NFC antenna 104, which extends in an axially limited portion in the area of the insertion aid 60 completely and within the wall of the shaft housing portion 34. The inserted end effector 18 has an RFID chip 106, which is read by the antenna 104 as it passes by. The end effector 18 may have any type of surgical working end. According to the disclosure, its characteristic, calibrated dimensions are recorded during its production and are, according to the disclosure, dimensions that have an influence on the position of the working point in space, such as the distance of the annular front surface/of the axial stop 76 to the working point 20, as well as its tolerance, as well as a concentricity of the working point 20 (cf. FIG. 8) or other information, such as a diameter, a surface roughness, a degree of sharpness and the like. The RFID chip 106 may, for example, carry the article number and the serial number of the end effector 18 as a minimum data set. When it passes the antenna 104, this information is read out and transmitted to the above-mentioned control unit. The type of end effector 18 is uniquely assigned to the article number and serial number in an attractable database. By mapping the article number, all other dimensions and tolerances of the inserted end effector 18 can then be applied and used to uniquely calculate the position of the new working point—without recalibration. Alternatively, the aforementioned data may of course already be stored on the RFID chip 106 itself and can be read out directly and transmitted to the control unit.

FIG. 17 shows a drive 550 with the housing 216 as shown in FIGS. 6 and 7, as well as with a motor 582 that is not lateral to the shaft housing portion 34, but is aligned with it. Accordingly, the motor 582 first has to be lifted off the housing 216 in order to replace the end effector 18. Subsequently, the shaft 70 is separated from the motor 582 and the new end effector 18 is inserted into the shaft housing portion 34 until the radial collar 72 of the end effector 18 axially stops with the axial stop of the distal coupling portion 32 (cf. central FIG. 17). Finally, the motor 582 is connected to the coupling 36.

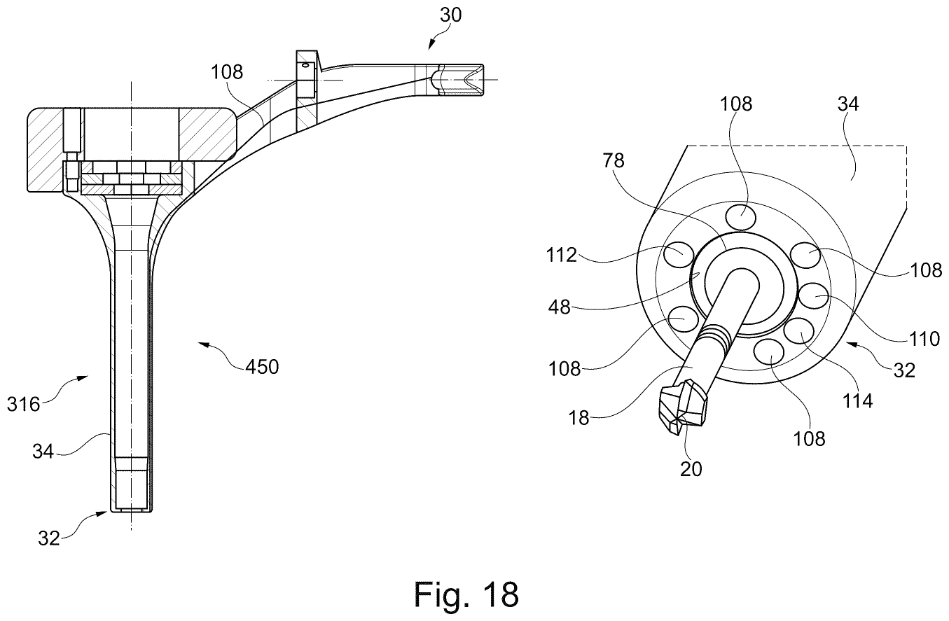

FIG. 18 shows the drive 450 with the housing 316 according to FIGS. 15, 16. The housing is configured with channels 108, 110, 112, 114, which extend from the proximal coupling portion 30 to the distal coupling portion 30 within the solid material of the housing 316 (cf. FIG. 18 left) and open distally (cf. FIG. 18 right) or are configured as blind channels. Due to the one-piece configuration of the housing 316 according to the disclosure, light, data, image data, coolant, coolant or the like may be arranged, transmitted or conveyed safely and without interference in the channels across this area. In the configuration example shown, for example, four optical fiber channels 108 for illuminating the surgical area, a sensor channel 110 of a sensor, via which the bearing forces of the end effector 18 may be detected, as well as a cooling channel 112 and a heating channel 114.

### LIST OF REFERENCE SIGNS

1 surgical robot
2, 4, 6, 8 segment
10 end segment
12 coupling arm
14 surgical instrument
16; 116; 216; 316 housing
18 end effector
20 working point
22, 24, 26, 28 coupling point
30, 230 proximal coupling portion
32 distal coupling portion
34 shaft housing portion
36 coupling
38 recess
40 coupling leg
42,44 coupling element
46 gearbox housing portion
48 outlet opening
50; 150; 250; 350; 450; 550 drive
52 handpiece
54 inlet opening
56 radial tapering
58 receiving space
60 insertion aid
62 axial stop
64 radial stop
66 axial coupling surface
68 lateral coupling surface
70 shaft
72 radial collar
74 working end
76 axial stop
78 centering collar
80 motor housing portion
82 drive motor
84 driveshaft
88 gearbox
90 drive spur gear
91 first gear stage
92 cogwheel
93 second gear stage
94 output spur gear
95 drive spur gear
96 coupling portion
98 coupling portion
99 output spur gear
100 coupling portion
101 coupling portion
103 output spur gear
104 NFC antenna
106 RFID chip
108 optical fiber channel
110 sensor channel
112 coolant channel
114 heating medium channel
H distance of axial stops
L distance of rotational axis to lateral stop
d diameter of radial stop

1-15. (canceled)

16. A medical instrument for a medical robot, the medical instrument comprising:
an end effector;
a support structure supportingly receiving the end effector;
a proximal coupling portion configured for coupling with a distal end segment of the medical robot;
a distal coupling portion configured for coupling with the end effector; and
a connecting component that extends at least between the proximal coupling portion and the distal coupling portion in one piece,
the end effector being exchangeable with a different end effector,
the connecting component establishing a coupling-free and/or tolerance-chain-free connection between the proximal coupling portion and the distal coupling portion,
the connecting component forming a housing having a shaft housing portion on which the distal coupling portion is configured,
the shaft housing portion having a proximal inlet opening through which the end effector is insertable, and
the shaft housing portion also having a distal outlet opening through which the end effector passes during an operation.

17. The medical instrument according to claim 16, wherein the connecting component forms a housing portion of the medical instrument.

18. The medical instrument according to claim 16, wherein the connecting component forms a frame structure which is arranged parallel to an instrument housing of the medical instrument and thus determines a relative position of the distal coupling portion to the proximal coupling portion.

19. The medical instrument according to claim 16, wherein the connecting component has high-precision shape tolerances, alignment tolerances and/or position tolerances, at least with regard to the proximal coupling portion and the distal coupling portion.

20. The medical instrument according to claim 16, wherein the proximal coupling portion and the distal coupling portion have contact planes or main axes having fixed parallel, angular or perpendicular alignment in relation to each other.

21. The medical instrument according to claim 16, wherein the shaft housing portion has a radial constriction or an inner radial collar, from which an axial stop of the distal coupling portion is formed for axial coupling with an axial stop of the end effector.

22. The medical instrument according to claim 16, wherein the shaft housing portion has an inner circumferential surface from which a radial stop of the distal coupling portion is formed for radial coupling with a radial stop of the end effector.

23. The medical instrument according to claim 16, further comprising a radial stop coaxial to the proximal inlet opening and/or an axial stop for a drive of the end effector or a drive train of the drive.

24. The medical instrument according to claim 16, further comprising a motor housing portion arranged laterally to the proximal inlet opening, the motor housing portion configured to accommodate a motor of a drive of the end effector at least in sections.

25. The medical instrument according to claim 16, further comprising a gearbox housing portion configured to receive a gearbox of a drive of the end effector at least in sections.

26. The medical instrument according to claim 25, further comprising a motor housing portion that is connected via the gearbox housing portion in alignment with the shaft housing portion.

27. The medical instrument according to claim 25, further comprising a motor housing portion that is laterally connected to the shaft housing portion.

28. The medical instrument according to claim 16, further comprising a sensor, by which at least one ID, characteristic properties and/or dimensions and tolerances of the end effector is readable.

29. The medical instrument according to claim 16, further comprising at least one tapering in the shaft housing portion, the at least one tapering being stepped in an insertion direction and configured for rotatable mounting of at least one drive element of at least one gear stage of a gearbox of a drive of the end effector.

30. The medical instrument according to claim 29, wherein:
output elements of several gear stages of a gearbox of a drive of the end effector are rotatably mounted in a region of the at least one tapering,
each output element has a passage recess having an internally circumferential coupling portion,
inner diameters of the passage recesses decrease in steps in an insertion direction,
each output element further comprising a set of end effectors that are exchangeable, each end effector having a shaft with an outer circumferential coupling portion ending at the same height in the insertion direction,
the outer circumferential coupling portions each have a unique pair of values of outside diameter and length such that the largest of the outside diameters is assigned the smallest length and the smallest of the outside diameters is assigned the largest length,
outside diameters between the largest of the outside diameters and the smallest of the outside diameters decrease in steps in the insertion direction, and
lengths located between the smallest length and the largest length increase in steps in the insertion direction.

31. The medical instrument according to claim 16, wherein the support structure is supportingly receiving the end effector and a drive of the end effector.

32. The medical instrument according to claim 16, wherein the connecting component extends at least between the proximal coupling portion and the distal coupling portion in one piece of material.

33. A support structure for a surgical/medical instrument for a surgical/medical robot, for supportingly receiving an end effector of the surgical/medical instrument, the support structure comprising:
a proximal coupling portion configured for coupling with a distal end segment of the surgical/medical robot;
a distal coupling portion configured for coupling with the end effector;
a connecting component extending at least between the proximal coupling portion and the distal coupling portion in one piece,
the connecting component establishing a coupling-free and/or tolerance-chain-free connection between the proximal coupling portion and the distal coupling portion,
the connecting component forming a housing having a shaft housing portion on which the distal coupling portion is configured,
the shaft housing portion having a proximal inlet opening through which the end effector is insertable, and
the shaft housing portion also having a distal outlet opening through which the end effector passes during an operation.

34. The support structure according to claim 33, wherein the support structure is configured for supportingly receiving the end effector and a drive of the end effector.

35. The support structure according to claim 33, wherein the connecting component extends at least between the proximal coupling portion and the distal coupling portion in one piece of material.
